(19) United States
(12) Patent Application Publication
(10) Pub. No.: US 2018/0185173 A1
(43) Pub. Date: Jul. 5, 2018

(54) PNEUMATIC DEVICE FOR ACTUATING ORGANS

(71) Applicants: SCUOLA SUPERIORE SANT'ANNA, PISA (IT); FONDAZIONE ISTITUTO ITALIANO DI TECNOLOGIA, GENOVA (IT)

(72) Inventors: Matteo CIANCHETTI, PISA (IT); Cecilia LASCHI, PISA (IT); Paolo DARIO, PISA (IT); Syed Taimoor Hassan SHAH, PESHAWAR (PK); Barbara MAZZOLAI, GENOVA (IT)

(21) Appl. No.: 15/739,629

(22) PCT Filed: Jun. 24, 2016
(86) PCT No.: PCT/IB16/53786

§ 371 Date: Dec. 22, 2017

(30) Foreign Application Priority Data
Jun. 26, 2015 (IT) ITUB20151717

(51) Int. Cl. A61F 2/68; B25J 9/14; F15B 15/10

(52) U.S. Cl. CPC A61F 2/68; A61F 2002/5066; F15B 15/103; B25J 9/142

(57) ABSTRACT

The device (1) takes up the basic structure and operation of the McKibben type artificial muscles and similar, in which is provided a hollow cylindrical chamber (2), of resilient material, which is inflated in the active phase (K). The chamber (2) interacts with a braided sleeve (3), consisting of threads (31, 32), almost inextensible, arranged in crossed helical paths having a characteristic angle of inclination β with respect to the longitudinal axis (X) of the device (1). The sleeve (3) is connected at the ends with two rigid head members (4A, 4B) provided to be mechanically connected to external bodies. The invention provides adjusting means (5), associated to said rigid head members (4A, 4B) adapted to vary their distance (D) at rest, so as to require a proportional and consistent change in the angle of inclination β: if the latter is exactly 54.7°, the device (1), in the active phase (K) stiffens without dimensional changes, if the angle β is greater an axial extension is obtained, if the angle β is smaller an axial contraction is obtained.

### TECHNICAL FIELD

The present invention relates to the technical sector concerning actuating devices able to impart movements to bodies.

In the cited technical field are present various types of such actuating devices, commonly used in mechanical applications and in robotics.

In particular, the invention relates to the devices known as artificial muscles or pneumatic muscles.

### BACKGROUND ART

In these devices, the mechanical work is not obtained from the movement of a piston in a cylinder but, conceptually, by exploiting the elastic deformation of rooms that are pressurized with air or other fluid.

The substantial difference lies in the fact that while a conventional pneumatic actuator has a ‘rigid’ behavior, that means it implements the movement only in the pre-determined mode, for example rectilinear, in an artificial muscle the behavior can vary according to the type of application and It may allow deviations of the thrust directions, or of shooting, because of external events, foreseen or unforeseen.

In the prior art document US2003/0205045A1, the artificial muscle comprises an elastic central cylindrical chamber, interposed between two head members, with the latter ones guided, for example, in the axial direction.

The side surface of said clastic central cylindrical chamber is circumscribed by an annular clastic chamber, whose internal wall is constituted by the same central chamber.

The two chambers are interconnect by a closed circuit pneumatic plant, comprising a connecting duct and a bi-directional pump.

By actuating the pump so that the fluid is removed from the cylindrical central chamber and placed in the annular chamber, a mutual approaching of the head members is obtained, that means a contraction of the artificial muscle.

By reversing the drive direction of the pump, of course, an opposite effect is obtained, with the mutual spacing of the head members, that means an extension of the artificial muscle.

The technique solution described is a relatively simple solution in terms of the operating principle, but in practice the need to have a closed circuit pneumatic system, with relative pump, determines both not negligible undesired dimensions and constructional complexity.

Similar devices are also known as McKibben muscles, in which is provided a cylindrical hollow chamber, tight, made of elastomeric material, adapted to be pressurized with air or other fluid by means of blowing, for example external.

On the outside of said hollow cylindrical chamber is coaxially disposed a braided sleeve, consisting of a first set of flexible and substantially inextensible wires, arranged each according to its own helical path of the same diameter and helix angle but different principles and by a second set of the same flexible and substantially inextensible wires, arranged according to respective helical paths symmetric to those of the above mentioned first set.

The said first and second sets of flexible wires, therefore, form the mesh of the above said sleeve, in which each of sad helical paths is inclined to a same predetermined angle with respect to the longitudinal axis of said device.

The device also comprises two rig d head members, provided at the corresponding ends of the braided sleeve, to which are attached both the head members of the respective threads and the cylindrical hollow chamber.

The rigid head members are adapted to be mechanically connected to external users means.

In an active phase of the device, the blow-in bodies bring pressure into the cylindrical hollow chamber, which interacts with the said braided sleeve: being the latter formed by inextensible threads, it can not follow in a homogeneous way the deformation undergone by the cylindrical hollow chamber, of elastic material.

From extensive studies, researches and experiments, it was found that the decisive factor to determine how the braided sleeve reacts, depends on the angle that each of the helical paths form with the longitudinal axis of said device when it is at rest; more precisely, it is seen that:
at an angle of exactly 54.7° C. it only gets a stiffening of the device, or muscle, so without any changes in length and lateral deformations;
with an angle smaller than 54.7° C. an axial contraction and an increase in the radial direction are obtained;
with an angle greater than 54.7° C. an axial extension and a reduction in the radial direction are obtained.

At the end of the pressuring action, the device returns to its original position.

In another prior art document, US005165323A, is described a pneumatic actuator similar to a McKibben muscle, comprising an elastic tubular clement, the ends of which are associated to two head members made of rigid material.

The elastic tubular element defines in its interior a chamber, while on the outside is wrapped in adhesion to a reinforcing mesh structure, consisting of threads of tract ion-resistant material, for example polyester, such as to be less yielding than the tubular elastic element.

The threads of the mesh, with the actuator at rest, have a predetermined angle of inclination with respect to the longitudinal axis of the actuator itself, which can be smaller than or greater than a characteristic angle, so-called of ‘stand-by’, that may have the previously indicated value.

Through a hole made in one of said head members, it is possible to pressurize the inner chamber; also in this case you get an approach or a withdrawal of the head members, that means a contraction or in extension of the actuator, in function of the angle of inclination originally established for the threads of the mesh, similarly to what previously said.

In the mentioned document it is claimed the presence of two sleeves which extend from the head members inside the chamber and telescopically mate, in order to guide the axial movement of the actuator.

Among the sleeves are shown sealing rings (O-rings) which isolate the chamber from surrounding possible losses; the volume of the chamber is reduced by the presence of the sleeves and requires a smaller air flow to be pressurized.

The drawback that most complains in the prior art devices classifiable as McKibben muscles concerns the fact that the pneumatic muscle response characteristics are laid down originally in function of the predetermined angle for the threads with respect to the longitudinal axis.

This means that a pneumatic muscle built for contraction will not stretch and vice versa, as well as a muscle that provides the angle exactly at 54.7° C. can not do other than stiffen without changing its extension.

In certain applications, where it is necessary to handle a generic user in opposite directions, therefore, we must combine two of said devices, with understandable constructive complications as well as increase of dimensions and costs.

Another drawback of the known devices concerns the fact that the predetermined angle of inclination not only determines the type of response, but also the extension or contraction stroke length, which can not be changed retrospectively.

Some researchers have experimented a technical solution to obtain actuators that could produce bi-directional movements: such solution provides an auxiliary chamber inside of the main one, adapted to be pressurized independently.

When the auxiliary chamber is not pressurized, the actuator is arranged for a shrinking movement as result of the pressurization of the main chamber; the volume occupied by the auxiliary chamber decreases request of air for the main chamber giving greater strength to the action.

When only the auxiliary chamber is pressurized, it expands axially causing a corresponding lengthening of the device.

The main drawback of this solution concerns the fact that the force exerted by the device is different in the two working conditions, with the one in extension considerably smaller than that in a contraction.

Additionally two power circuits are needed, or at least exchange valves to divert the flow of compressed air towards one or the other chamber.

All this results, inevitably, in an increase of the overall dimensions and weights.

Finally, also the limitation of other solutions in which the characteristics are predefined and not editable remains.

### SUMMARY OF THE INVENTION

The object of the present invention is therefore to propose a pneumatic device for the actuation of organs, which is similar to the working principles of McKibben type artificial muscles and similar, but shaped so as to obviate the limits of the known embodiments, in particular as regards the ability to be configured at will at any time, to obtain an active phase in the contraction, or in extension or only stiffening.

An another object of the invention resides in the desire to provide a device that offers the above mentioned characteristics by means of constructive solutions at the same time simple, effective and reliable.

Still another object of the invention aims to obtain a device of compact size and regular shape, so as to be easily installed in all applications without special care.

A further object of the invention is to propose a device which can be built in series with contained costs, by using materials, machineries and equipments of common use in the industry.

These and other objects are fully achieved by a pneumatic device for the actuation of organs, in which are provided: a cylindrical hollow chamber, tight, made of elastomeric material, adapted to be pressurized with air or other fluid; a braided sleeve, coaxially disposed outside said hollow cylindrical chamber and constituted by a first set of flexible and substantially inextensible wires, arranged each one according to its own helical path of the same diameter and helix angle but different principles, and by a second set of the same flexible and substantially inextensible wires, arranged according to respective helical paths symmetric to those of the above mentioned first set, with said first and second set of flexible threads adapted to form the meshes of said net, in which each of said helical paths is inclined of a same predetermined angle with respect to the longitudinal axis of above said device; two rigid head members, provided to the corresponding ends of said braided sleeve, to which are fixed the respective ends of the mentioned wires, with the same rigid head members provided to be mechanically connected to external users means; blow in-organs, adapted to pressurize or depressurize said cylindrical hollow chamber, respectively in an active phase, or in a resting phase of the said device, in the first of which said hollow cylindrical chamber interacts with the above mentioned braided sleeve to get an extension, or a contraction, or a stiffening of the same device, respectively, if the mentioned angle of inclination of each helical path is, originally, greater than, smaller than or equal to a predetermined value, with said device comprising: adjusting means, associated with said rigid head members, adapted to modify, increasing or decreasing, the distance originally provided between the same rigid head members in said stand-by-phase of the device, so as to require a proportional and coherent variation of the angle of inclination of each helical path in said braided sleeve, such that said device, brought in its mentioned active phase, switches its response characteristics, determining for itself an axial extension, or an axial contraction, or a stiffening, respectively if, with a given distance in stand-by between the said rigid head members, the said angle of inclination is greater than, smaller than or equal to said predetermined value.

### DESCRIPTION OF DRAWINGS

The characteristics of the invention will be made evident in the following description of preferred embodiments of the pneumatic device for the actuation of organs in the object, in accordance with the contents of the claims and with the help of the enclosed drawings, in which:

FIG. 1 shows a first embodiment of the device in object;

FIG. 2 shows an axial section of the device of FIG. 1;

FIG. 3 shows an axial section of a second embodiment of the device;

FIG. 4 shows an axial section of a third embodiment of the device;

FIGS. 5A, 5B, 5C schematically show an embodiment arranged with two devices parallel matched, having the same initial adjustment, in different operative phases;

FIGS. 6A, 6B schematically show an embodiment arranged similar to the previous one with the two devices having different initial adjustment, in two operating steps;

FIGS. 7A, 7B schematically show an embodiment arranged similar to the previous one with the two devices yet having identical initial adjustment, opposite to that of FIG. 5A, in two operating steps;

FIGS. 8A, 8B schematically show an embodiment in which the device is arranged to move a control lever in two opposite directions;

FIG. 9 show the device applied to an orthopedic device;

FIG. 10 schematically show an arrangement that provides multiple devices adapted to interact to obtain complex movements.

### DETAILED DESCRIPTION OF PREFERRED EMBODIMENTS

In the above listed figures, it has been indicated with reference 1, the pneumatic device for the actuating organs, object of the present invention.

The device 1 follows (he general principles of operation of the McKibben type artificial muscles and similar, of which it is said in the introduction, and provides, in an itself known manner
a hollow cylindrical chamber 2, tight, made of elastomeric material, adapted to be pressurized with air or other fluid by external means of blowing, not illustrated, constituted, for example, by a compressed air supply system;
a braided sleeve 3, coaxially arranged outside said hollow cylindrical chamber 2 and constituted of a first set of threads 31 flexible and substantially inextensible, arranged each according to its own helical path of the same diameter and helix angle b but different principles, and by a second set of identical threads 32 flexible and substantially inextensible, arranged according to respective helical paths symmetrical to those of the above mentioned first set, with said first and second set of flexible threads 31, 32 adapted to form the meshes of said net 3, in which each of said helical paths is inclined of a same predetermined angle b with respect to the longitudinal axis X of the above said device 1;
two rigid head members 4A, 4B provided at the corresponding ends of said braided sleeve 3, to which are fixed the respective ends of the mentioned threads 31, 32, with the same rigid head members 4A, 4B provided to be mechanically connected to external users means, not illustrated.

The blow-in bodies are adapted to pressurize or depressurize said hollow cylindrical chamber 2, respectively in an active phase K or in a stand-by phase W of the mentioned device 1, in the first of which said hollow cylindrical chamber 2 interacts with the said braided sleeve 3 to obtain an extension or a contraction, or a stiffening of the same device 1, respectively, if the mentioned angle of inclination b of each helical path is, originally, greater than, less than or equal to a predetermined value. The above mentioned predetermined value corresponds to an angle of 54.7° C. which is a theoretical limit that has been verified and experimentally validated.

According to the invention, the device 1 comprises adjusting means 5, associated to said rigid head members 4A, 4B and adapted to modify, increasing or decreasing, the distance D originally provided between the same rigid head members 4A, 4B in said phase of stand-by W of the device 1, so as to require a proportional and coherent variation of the angle of inclination b of each helical path in the above said braided sleeve 3.

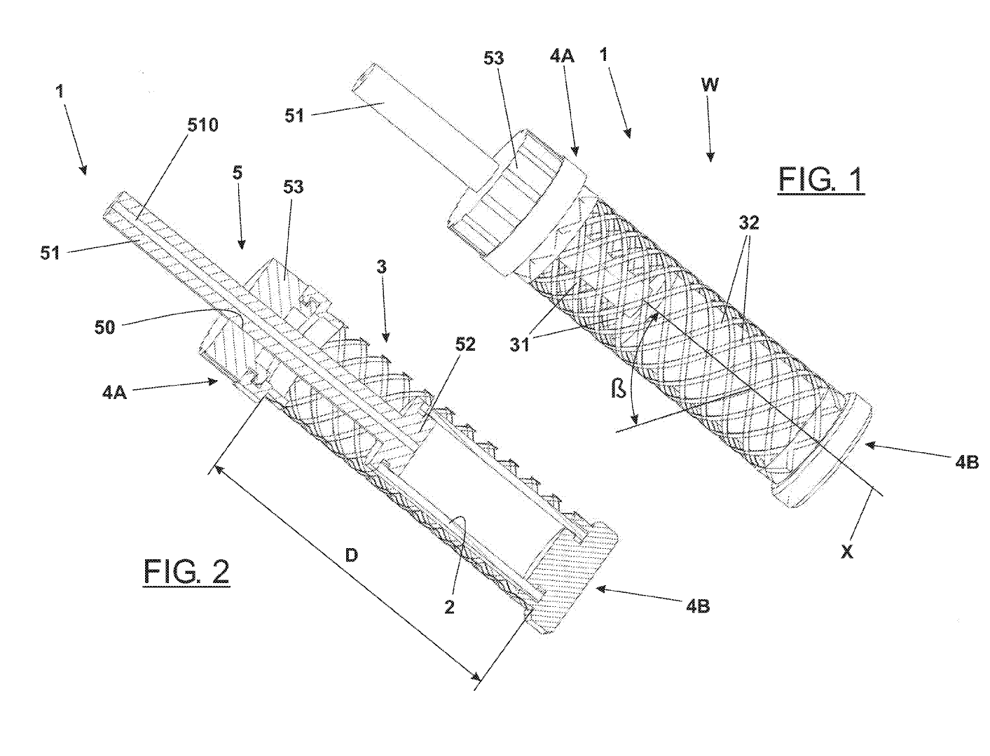

In a first embodiment of the device 1, illustrated in FIGS. 1 and 2, said adjusting means 5 comprise:
an axial through hole 50 made in one of said rigid head members, for example the head 4A;
a stem 51, inserted into the axial through hole 50 and extended on both sides with respect to the mentioned rigid head IA;
a end plate 52, made integral to the end of the stem 51 located in an intermediate position between said rigid head members 4A, 4B inside said braided sleeve 3, said end plate 52 being placed in closure of a corresponding end of said hollow cylindrical chamber 2;
operating means 53, interposed between the axial through hole 50 and the stem 51, adapted to allow axial translations of the respective rigid head 4A with respect to the stem 51 itself, thereby increasing or decreasing the distance D with the remaining rigid head 4B and the length of the braided sleeve 3.

In a preferred constructional solution, said axial through hole 50 and the stem 51 are threaded and mutually engaged, and the mentioned operating means 53 are constituted by a rotating pawl provided in said rigid head 4A and bearing the axial threaded through hole 50.

The pawl 53 can be rotated in one direction or the other to determine the above said offsets of the corresponding rigid head 4A along the stem 51; the position reached from time to time is automatically stabilized by the above mentioned threaded coupling between the through hole 50 and the stem 51.

In the embodiment referred to in FIGS. 1 and 2, the stem 51 is provided with an axial passing through conduit 510, adapted to connect the said blowing means with the hollow cylindrical chamber 2; alternatively it is possible to provide a passing through duct made in the remaining rigid head 4B.

With the rotation of the pawl 53, and the consequent variation of the distance D between the rigid head members 4A, 4B, is determined the initial value of the said angle of inclination b of each helical path, so that the device 1, brought in its mentioned active stage K, switches its response characteristics, according to the following correlations:
with an angle b of exactly 54.7° C. it only gets a stiffening of the device 1 so without changes in length of the same and without lateral deformations;
with an angle b 1 less than 54.7° C., imposed increasing the distance D, it gets an axial contraction and an increase in the radial direction;
with an angle b 2 greater than 54.7° C., imposed by decreasing the distance D, it gels an axial extension and a reduction in the radial direction.

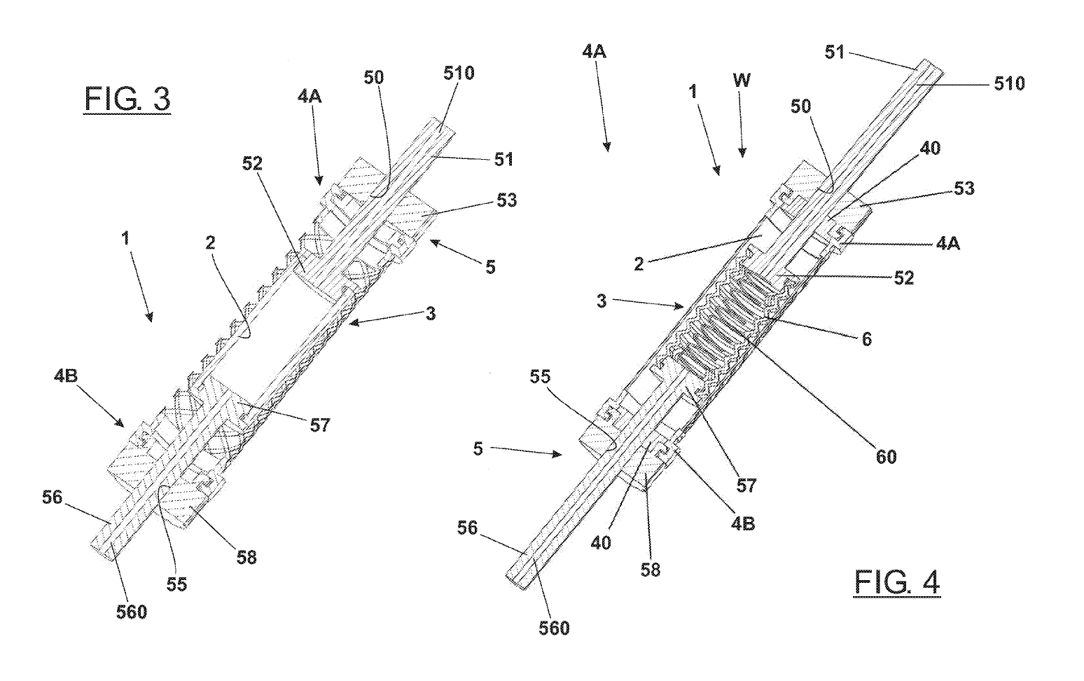

In a second embodiment of the device 1, illustrated in FIG. 3, said adjusting means 5 comprise:
a first and a second axial through hole 50, 55 respectively, made in said rigid head members 4A, 4B;
a first and a second stem 51, 56, respectively inserted into said first and second axial through hole 50, 55, with each of said stem 51, 56 extending both sides with respect to the corresponding rigid head 4A, 4B;
a first and a second end plate 52, 57, each of which is made integral with the end of the respective stem 51, 56 located in an intermediate position between the rigid head members 4A, 4B inside the braided sleeve 3, said first and second end plate 52, 57 being in closing position of the corresponding ends of said hollow cylindrical chamber 2, situated in an intermediate position with respect to the braided sleeve 3;
first and second operating means 53, 58, interposed respectively between the said first hole 50 and first stem 51 and between the said second hole 55 and second stem 56, adapted to allow axial translations of the respective rigid head 4A, 4B with respect to the corresponding stem 51, 56, thereby increasing or decreasing the distance D with the remaining rigid head 4A, 4B, as well as the length of said braided sleeve 3.

In a preferred constructive solution, in compliance with what already provided for said first form of embodiment, the first axial through hole 50 and the first stem 51 are threaded and mutually engaged, as well as the second axial through hole 55 and the second stem 56.

The first operating means 53 are constituted by a first rotatable pawl provided in the relative rigid head 4A and bearing the first threaded axial through hole 50; similarly, the second operating means 58 are constituted by a second rotatable pawl, for example, equal to the first, provided for in the remaining rigid head 4b and bearing the second threaded axial through hole 55.

Each pawl 53, 58 can be rotated in one direction or the other, independently, to determine axial translations of the corresponding rigid head 4A, 4B with respect to the other.

The threaded couplings between the through holes 50, 55 and respective stems 51, 56 stabilize the position which is time after time achieved by each rigid head 4A, 4B.

In the embodiment of FIG. 3, both items 51, 56 are provided with an axial through conduit 510, 560, each adapted to connect the said blowing means with the hollow cylindrical chamber 2; alternatively it is possible to provide only one passing through duct in one of the two stems 51, 56.

As already explained with regard to the first embodiment, with the rotation of the first and/or of the second pawl 53, 58 and the consequent variation of the distance D between the rigid head members 4A, 4B, it is determined the initial value of the mentioned inclination angle b of each helical path, so that the device 1, taken in its mentioned active phase K, switches its response characteristics, according to the correlations previously set out.

The described second embodiment of the device 1 extends the calibration interval granted to said adjusting means 5, thereby increasing the versatility of the device 1 itself.

In a third embodiment of the device 1, illustrated in FIG. 4, said hollow cylindrical chamber 2 is associated fitting inside said braided sleeve 3 and has the same axial development of this.

As a constructional variant, it is possible to predict that the hollow cylindrical chamber 2 is incorporated in the sleeve 3 itself, though remaining the structural differences of the one and the other which make the first one clastic and the second one formed by substantially inextensible wires.

The corresponding adjusting means 5 comprise:
a first and a second axial through hole 50, 55, respectively made in said rigid head members 4A, 4B;
a first and a second stem 51, 56, respectively inserted into said first and second axial through hole 50, 55, with each of said stems 51, 56 extending both sides with respect to the corresponding rigid head 4A, 4B;
a first and a second end plate 52, 57, each of which is made integral with the end of the respective stem 51, 56 located in an intermediate position between the rigid head members 4A, 4B inside the braided sleeve 3 and the hollow cylindrical chamber 2, said first and second end plate 52, 57 being connected to the opposite ends of a bellows 6 of clastic material, located in an intermediate position with respect to said braided sleeve 3 and hollow cylindrical chamber 2;
at least one elastic member 60, housed in said bellows 6 and interposed between the said bottoms 52, 57, adapted to resiliently push the same in mutual removal;
first and second operating members 53, 58, interposed respectively between the said first hole 50 and first stem 51 and between the said second hole 55 and second stem 56, adapted to allow axial translations of the respective rigid head 4A, 4B with respect to the corresponding stem 51, 56, thereby increasing or decreasing the distance D with the remaining rigid head 4A, 4B, as well as the length of said braided sleeve 3.

In a preferred constructive solution, in compliance with what already provided for the previous forms of embodiment, the first axial through hole 50 and the first stem 51 are threaded and mutually engaged, as well as the second axial through hole 55 and the second stem 56.

The respective first and second operating members 53, 58 are similarly constituted by a first and a second rotatable pawl provided in the relative rigid head members 4A, 4B adapted to be rotated in one direction or another, in an independent manner, to determine such axial offsets of the corresponding rigid head 4A, 4B with respect to the other.

Also in this case, the threaded couplings between the through holes 50, 55 and the respective stems 51, 56 stabilize the position reached time after time by each rigid head 4A, 4B.

The elastic member 60 inside the bellows 6 stabilizes the structure of the device 1 during the calibration operations of the angle of inclination b.

In the embodiment of FIG. 4, both stems 51, 56 are provided with an axial through conduit 510, 560, each adapted to connect the said blowing means with the interior of the bellows 6, which in turn has openings (not shown in detail) which allow the introduced pressurized air flow to merge inside the hollow cylindrical chamber 2; alternatively it is possible to provide only one passing through duct in one of the two stems 51, 56.

In each of the rigid head members 4A, 4B is provided a tight o ring 40 which engages the respective stem 51, 56 and prevents the compressed air to escape from the hollow cylindrical chamber 2 through the through holes 50, 55.

During the active step K of the device 1, the bellows 6 as well as the elastic member 60 are compressed or elongated in agreement with the contraction or extension of the device 1 itself.

In the figures from 5A to 7B is illustrated schematically an application with two devices 1 matched parallel, mechanically connected to a fixed upper support 70 and to a lower bar 80.

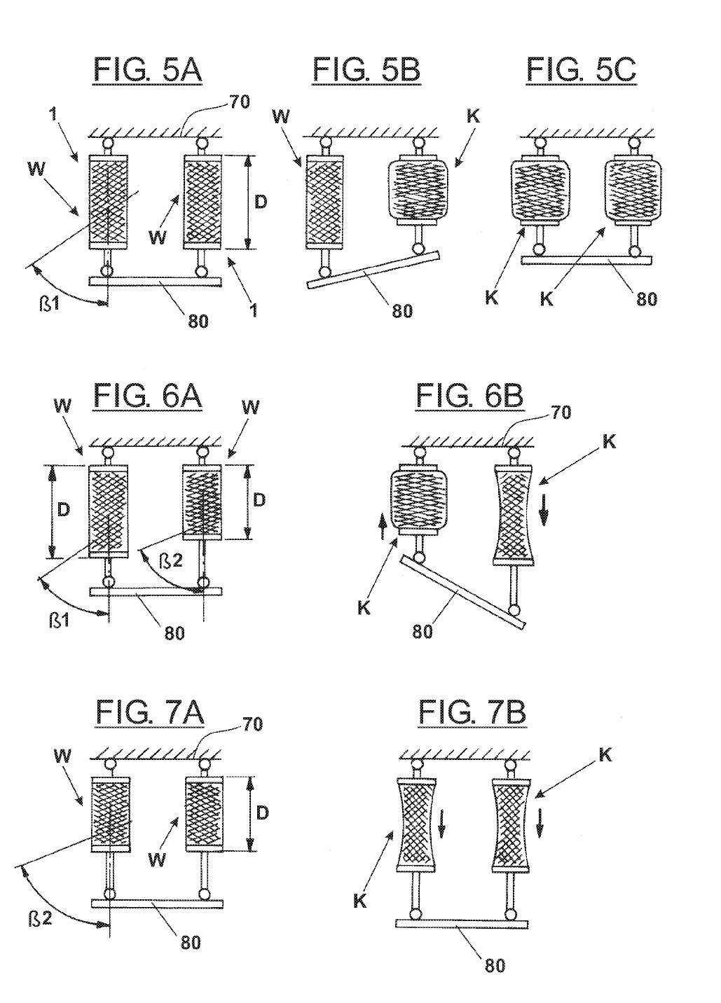

In FIG. 5A the devices 1 have the same initial adjustment, with an angle of inclination b1 of the helical paths of 54.7° C., imposed by increasing the distance D between the rigid head members 4A, 4B; both the devices 1 are then prepared for an axial contraction and an increase in the radial direction.

In FIG. 5B only the device 1 on the right is in its active phase K, for which the bar 80 is sloped, while with the activation of both devices 1, as shown in FIG. 5C, the bar 80 is again horizontal but at a level higher than that one of departure.

In FIG. 6A the devices 1 have different initial adjustment: the left one presents an angle of inclination of the helical paths b1 smaller than 54.7° C., therefore predisposed to the contraction, while the right one has an angle b2 greater than 54.7° C., then predisposed to the extension.

FIG. 6B shows how the bar 80 is sloped (with higher angle with respect to the position taken in FIG. 5B) with the simultaneous activation of the two devices 1.

In FIG. 7A the devices 1 have the same initial adjustment, with an angle b2 greater than 54.7° C., then both are predisposed to the extension, as shown in FIG. 7B, in which the bar 80 moves parallel to the initial position and places itself at a lower level.

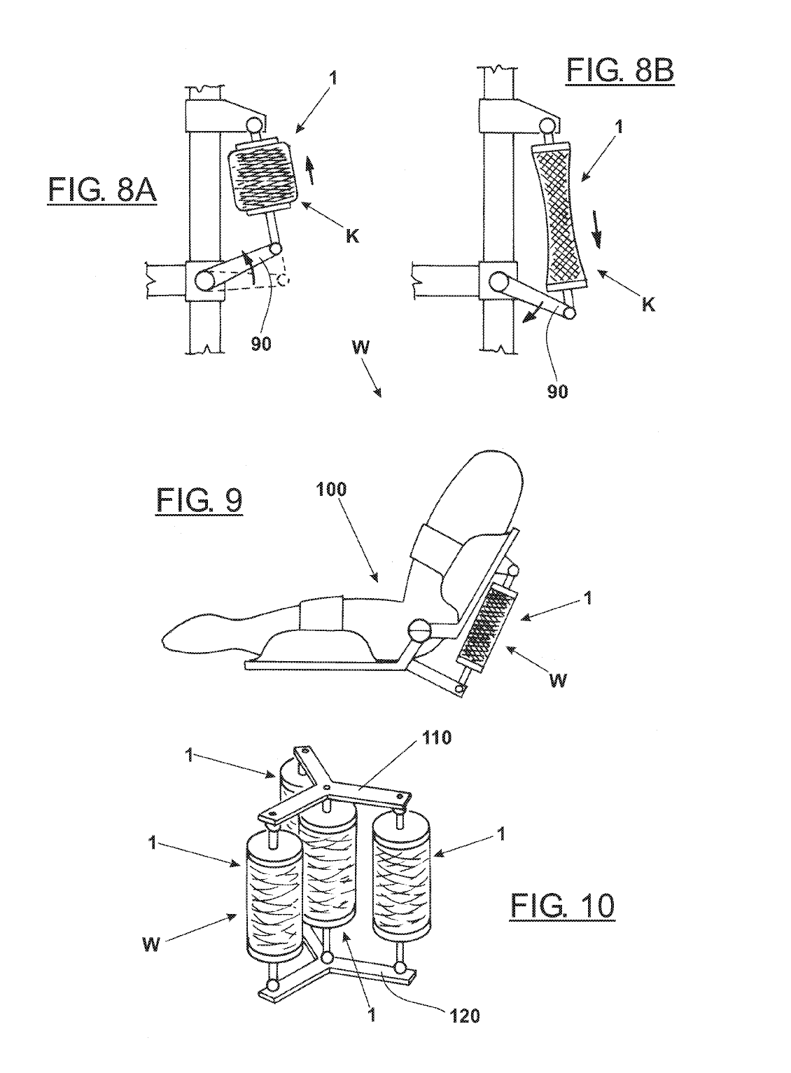

In FIGS. 8A, 8B is illustrated schematically an application in which the device 1 is set up to move a control bar 90, for example associated with a flow diverter valve.

As it can be seen, depending on the initial adjustment of the angle b the bar 90 can be driven in two opposite directions.

In FIG. 9 is illustrated, again as a way of example, another possible application in which the device 1 is associated with an orthopedic device 100 for the rehabilitation of an upper limb; according to the usual adjustment of the angle b it will be possible to get the desired bending movement.

In FIG. 10 is illustrated a further possible application that provides the presence of four devices 1, parallel to each other and radially arranged.

The devices 1 are connected at the top to a first supporting cross 110, and at the bottom to a second supporting cross 120, for example equal to the first.

Depending on how the angles b of each device 1 are adjusted, and on the activation mode of these, it can be obtained different orientations between said supporting crosses 110, 120, similar to those granted by a ball joint, but with the application of thrust forces; it understandable how similar applications are interesting in the field of robotics and/or bio-robotics.

From the previous description emerge in the evidence the peculiar characteristics of the proposed device, which incorporates the operating principles of McKibben type artificial muscle and similar, but it introduces original construction solutions that allow it to be configured at will at any time, to obtain an active phase in the contraction, or in extension or only of stiffening.

With this important prerogative they are totally exceeded the limits of the known embodiments and it is greatly simplified any practical application of the device.

It should be highlighted how the advantageous qualities of the proposed device are obtained with techniques at the same time simple, effective and reliable which allow besides to have a compact size and regular shape.

All the constructional solutions adopted meet the requirements that allow an easy serial production of the device with contained costs, as it could be used materials, machinery and equipment of common use in the industry.

As intuitively understandable, the device can be employed in various sectors of industry, automation, robotics, bio-medical, just to name a few.

It is understood however that what above said has value of example and not limiting, therefore any modifications of detail that may be necessary to make for technical and/or functional reasons, are considered from now as remaining within the protective scope defined by the claims below.

1-10. (canceled)

11. A pneumatic actuating device for the actuation of organs, comprising:
a chamber, wherein the chamber is an airtight, hollow cylindrical member made of an elastomeric material and is adapted to be pressurized with air or other fluid;
a sleeve, wherein the sleeve is braided, wherein the sleeve is disposed coaxially outside the chamber and constituted by a first series of flexible and substantially inextensible threads, arranged each according to its own helical path of the same diameter and helix angle but different start points, and by a second series of identical flexible and substantially inextensible threads, arranged according to respective helical paths symmetrical to those of the above mentioned first series, with the first and second set of flexible threads adapted to form the meshes of the sleeve, in which each of the helical paths is inclined with a same predetermined angle with respect to the longitudinal axis of the device;
two rigid head members, provided at the corresponding ends of the sleeve, to which are fixed at the respective start points of the mentioned threads, with the same rigid head members adapted to be mechanically connected to external utilizing means;
blowing means, adapted to pressurize or depressurize the chamber, respectively in an active phase or in a stand-by phase of the device, in the first of which the chamber interacts with the sleeve to obtain an extension or a contraction, or a stiffening of the same device, respectively, if the aforementioned angle of inclination of each helical path is, originally, greater than, less than or equal to a predetermined value; and
adjusting means, associated to the rigid head members, adapted to modify, by increasing or decreasing, the distance originally provided between the same rigid head members in the stand-by phase of the device, so as to cause a proportional and coherent variation of the angle of inclination of each helix in the sleeve, such that the response of the device when it is pressurized in its mentioned active phase, switches with determining an axial extension, or an axial contraction, or a stiffening, respectively, if, with a given distance in the stand-by phase between the rigid head members, the angle of inclination is greater than, less than or equal to the predetermined value.

12. The device according to claim 11, wherein the adjusting means comprise:
an axial through hole realized in one of the rigid head members;
a stem, inserted in the axial through hole extended form both sides of the rigid head member;
an end plate, made integral with the end of the stem located in an intermediate position between the rigid head members, inside the sleeve, the end plate being placed to close a corresponding end of the chamber; and
operating means, interposed between the axial through hole and the stem, adapted to allow axial offsets of the mentioned rigid head member with respect to the stem itself, thereby increasing or decreasing the distance with the remaining rigid head member, as well as the length of the above the sleeve, with the same operating means also adapted to lock the position set for the rigid head member.

13. The device according to claim 11, wherein the adjusting means comprise:
a first and a second axial through hole, respectively, made in the rigid head members;
a first and a second stem, respectively, inserted in the first and second axial through holes, with each of the stems extending both sides from the corresponding rigid head member;
a first and a second end plate, each of which made integral with the end of the respective stem located in an intermediate position between the rigid head members, inside the sleeve, the first and second end plate being arranged for closing the corresponding ends of the chamber, placed in an intermediate position with respect to the sleeve;
first and second operating members, interposed respectively between the first axial through hole and first stem and between the second axial through hole and second stem, adapted to allow axial offsets of the respective rigid head member compared to the corresponding stem, thereby increasing or decreasing the distance with the remaining rigid head member, as well as the length of the above the sleeve, with the same first and second operating members also adapted to lock the position set for the corresponding rigid head member.

14. The device according to claim 11, wherein the chamber fits inside the sleeve and it has the same axial extension, wherein the adjusting means comprises:
first and second axial through hole, respectively, made in the rigid head members;
first and second stems, respectively, inserted in the first and second axial through holes, with each of the stems extending both sides from the corresponding rigid head member;
first and second end plates, each of which is made integral with the end of the respective stem located in an intermediate position between the rigid head members, inside the sleeve and the chamber, the first and second end plates being connected to the opposite ends of a bellows of resilient material, located in an intermediate position with respect to the sleeve and the chamber;
at least one elastic member, housed in the bellows and interposed between the end plates, adapted to elastically push away one from the other the end plates; and
first and second operating members, respectively interposed between the first hole and first stem and between the second hole and second stem, adapted to allow axial offsets of the respective rigid head member compared to the corresponding stem, thereby increasing or decreasing the distance with the remaining rigid head member, as well as the length of the above the sleeve, with the same first and second operating members also adapted to lock the position set for the corresponding rigid head member.

15. The device according to claim 12, wherein the axial through hole and stem are threaded and mutually engaged, wherein the operating means comprise a rotatable pawl provided in the rigid head member and bearing the threaded axial through hole, with the pawl adapted to be rotated in one direction or in the other to determine the axial translations of the rigid head member along the stem, and wherein the threaded coupling between the through hole and stem is adapted to stabilize the position time after time reached by the same rigid head member.

16. The device according to claim 12, wherein the stem is provided with an axial through conduit, adapted to connect the blowing means with the chamber, the blowing means being provided outside the device and adapted to pressurize or depressurize the latter.

17. The device according to claim 15, wherein the stem is provided with an axial through conduit, adapted to connect the blowing means with the chamber, the blowing means being provided outside the device and adapted to pressurize or depressurize the latter.

18. The device according to claim 13, wherein the first and second axial through holes and first and second stems are threaded and mutually engaged, by the operating means consisting of first and second rotatable pawls provided in the relative rigid head members and bearing the first and second threaded axial through hole, with the first and second pawls adapted to be rotated in a direction or the other, in an independent manner, to determine the axial offsets of the corresponding rigid head member relative to each other along the stems, and in that the threaded couplings between the through holes and relative stems are adapted to stabilize the position which is reached time after time by each rigid head member.

19. The device according to claim 14, wherein the first and second axial through holes and first and second stems are threaded and mutually engaged, by the operating means consisting of first and second rotatable pawls provided in the relative rigid head members and bearing the first and second threaded axial through holes, with the first and second pawls adapted to be rotated in a direction or the other, in an independent manner, to determine the axial offsets of the corresponding rigid head members relative to each other along the stems, and in that the threaded couplings between the through holes and relative stems are adapted to stabilize the position which is reached time after time by each rigid head member.

20. The device according to claim 13, wherein at least one of the stems is provided with an axial through conduit, adapted to connect the blowing means with the chamber, the blowing means being provided on the outside of the mentioned device and adapted to pressurize or depressurize the latter.

21. The device according to claim 14, wherein at least one of the stems is provided with a axial through conduit, adapted to connect the blowing means, provided outside of the device to send a fluid under pressure, with the interior of the bellows, which is affected by openings for the passage of air under pressure towards the interior of the chamber intended to be pressurized.

22. The device according to claim 14, wherein the chamber is embedded in the sleeve, with the first being elastic and with the second defined by substantially inextensible threads.
